(19) United States
(12) Patent Application Publication
(10) Pub. No.: US 2025/0273312 A1
(43) Pub. Date: Aug. 28, 2025

(54) SYSTEM AND METHOD FOR IMMUNOSUPPRESSANT DOSAGE

(71) Applicants: THE REGENTS OF THE UNIVERSITY OF CALIFORNIA, Oakland, CA (US); UNIVERSITY OF FLORIDA RESEARCH FOUNDATION, INCORPORATED, Gainesville, FL (US)

(72) Inventors: Chih-Ming HO, Los Angeles, CA (US); Jeffrey KHONG, Anaheim, CA (US); Megan LEE, Torrance, CA (US); Ali ZARRINPAR, Gainesville, FL (US); Viki Eunbi KIM, Philadelphia, PA (US); Curtis WARREN, Gainesville, FL (US)

(73) Assignees: THE REGENTS OF THE UNIVERSITY OF CALIFORNIA, Oakland, CA (US); UNIVERSITY OF FLORIDA RESEARCH FOUNDATION, INCORPORATED, Gainesville, FL (US)

(21) Appl. No.: 18/684,546

(22) PCT Filed: Aug. 16, 2022
(86) PCT No.: PCT/US22/75033

§ 371 Date: Feb. 16, 2024

Related U.S. Application Data
(60) Provisional application No. 63/234,124, filed on Aug. 17, 2021.

(51) Int. Cl. G16H 20/10

(52) U.S. Cl. CPC G16H 20/10

(57) ABSTRACT

The present disclosure includes systems and methods for calculating a dosage of one or more medications. Some aspects include determining a dosage for a patient after an organ transplant, at least by determining a first dosage of a drug given to a patient on a first day, determining a first serum level of the patient associated with the first day, calculating a second dosage of the drug based on a first ratio between the first dosage and the first serum level, and generating a first output based on the second dosage. Some aspects include determining a fourth dosage of the drug based on: a second ratio between the second dosage and a second serum level and a third ratio between a third dosage and a third serum level. Some aspects include generating a second output based on the fourth dosage, the fourth dosage associated with a fourth time period.

### CROSS-REFERENCE TO RELATED PATENT APPLICATIONS

This application claims the benefit of priority of U.S. Provisional Patent Application No. 63/234,124 filed on Aug. 17, 2021 and titled “SYSTEM AND METHOD FOR IMMUNOSUPPRESSANT DOSAGE,” which is hereby incorporated by reference in its entirety.

### GOVERNMENT LICENSE RIGHTS

This invention was made with government support under Grant Number TR002087 and DK116140, awarded by the National Institutes of Health. The government has certain rights in the invention.

### TECHNICAL FIELD

Aspects of the present disclosure relate generally to apparatus, systems, and methods for determining a dosage of one or more medications and, more particularly but without limitation, to a system for determining an immunosuppressant dosage or serum level, such as a tacrolimus serum level, for a patient after an organ transplant.

### BACKGROUND

Advances in science and medical technology have increased the effectiveness of organ transplants and, as a result, organ transplant recipients may enjoy an extended life, improved quality of life, or increased mobility. However, an organ transplant, such as a transplant of the liver, kidney, heart, lung, and the like, is not without risk or complication. For example, one concern associated with an organ transplant is the post-operative risk of rejection of the organ by a recipient's body. To diminish this risk, one or more immunosuppressants, such as tacrolimus, mycophenolate and/or prednisone, may be used to diminish the body's immune response and mitigate the body's natural reaction to a foreign object—i.e., the transplanted organ.

The chance of organ rejection is the highest in the days immediately following a transplant. To promote organ acceptance, the concentration of the immunosuppressant drug in the patient's blood stream—or immunosuppressant serum level—should be maintained within a target range, typically measured in nanograms of immunosuppressant per milliliter of blood (ng/mL). Under-dosing of the immunosuppressing drug may result in underimmunosuppression and acute rejection of the new organ. Alternatively, over-dosing the immunosuppressing drug can lead to considerable neuro- and nephrotoxicity resulting in seizures, kidney failure, or other side effects.

However, maintaining the immunosuppressant serum level can be very difficult in practice. One such reason is due to the fact that target ranges of the serum levels are often very narrow-within 2-3 ng/ml-which requires precise and accurate dosing. Additionally, the serum levels may be affected by several factors outside of immunosuppressant dosage, including the type and dosage of other medications, liver function, kidney function, metabolism, intra- and interpatient variability, pharmacokinetics, and other physiological conditions, as illustrative, non-limiting examples. The important task of getting tacrolimus blood levels to therapeutic levels without either under- or overdosing is managed by the clinical team on a daily basis. This team typically includes transplant pharmacists and transplant physicians who together take into account a combination of clinical and physiological factors such as organ function, recovery of bowel function and absorption, as well as the various other concomitant medications the patient is receiving. This complex system is difficult to navigate and has not lent itself well to predictive algorithms.

One conventional method used in clinical practice is dose titration in response to daily measurements. This method of determining immunosuppressant dosage uses clinically measured serum drug level of a patient for the previous three days to select an immunosuppressant dosage for the following day after the three days. This conventional method works well in determining a dosage with a stable patient and three days of data; however, accuracy of the conventional method may decrease with patient instability as the determination depends on one or more physiological conditions, which may be unstable with changes in the post-transplant treatment regimen, such as the administration of new medications, hemodialysis, changes in dosages of other medications, or the like. To illustrate, during the one or more of the initial post-transplant days, the markers of liver function or injury (e.g., aspartate transaminase (AST), alanine transaminase (ALT)), and kidney function (e.g., glomerular filtration rate (GFR)) are not constant. Accordingly, three days of patient stability may not occur until several days or weeks after organ transplant. For example, the AST and ALT levels can vary dramatically for several days after a transplant, such as a change from several thousand units per liter (IU/L) to less than ten IU/L over a few days.

Such unpredictability makes calculating an immunosuppressant dose difficult and often requires experienced treating physicians to determine a suitable immunosuppressant dosage for each day after the operation. Additionally, calculating an immunosuppressant dosage may also be challenging immediately following transplant when patient physiological conditions are unstable. Accordingly, determining an immunosuppressant dosage continues to present a challenge immediately following a transplant when one or more physiological conditions may be unstable or unreliable.

### SUMMARY

The present disclosure describes systems, devices, and methods for determining a dosage of a medication. For example, the present disclosure describes a method for determining an immunosuppressant dosage for the recipient of an organ transplant. In some aspects, the present disclosure provides systems and methods for determining a drug dosage for, or after, the one or more time periods or one or more days immediately following the organ transplant. In some implementations, the methods include obtaining an indication of a first serum level of the patient associated with a first dosage of an immunosuppressant drug administered to a patient at a first time. Some methods may further include generating a first output that indicates a second dosage of the immunosuppressant drug derived from (e.g., calculated) based on a first immunosuppressant ratio. The first immunosuppressant ratio may be based on the first dosage and the first serum level. In some of the forgoing methods, the first immunosuppressant ratio is a ratio of the first dosage and the first serum level. The second dosage may be associated with a second time that is subsequent to the first time. Some methods may include updating medical data during, or prior to, generating the first output. In some implementations, the medical data may include a patient profile, dosage history, serum level history, or other information or measurements related to the patient.

In some aspects of the present methods, the first serum level includes an amount of immunosuppressant drug in the patient's blood stream. In some methods, obtaining the indication includes receiving the indication, such as receiving an input or a data message at a device. In some implementations, the first time is associated with a first time period, such as a time period less than or equal to a twenty-four hour period. Additionally, the second time may be associated with a second time period, such as a time period less than or equal to a twenty-four hour period. In some implementations, the first time is associated with a day and the second time is associated with a next day after the day. The day may be the day of an organ transplant operation, a day after the organ transplant operation, or a subsequent day following the organ transplant operation. To illustrate, the day may be a twenty-four hour period that occurs within five days immediately following an organ transplant operation.

Some aspects of the present methods include receiving an indication of the first dosage of the immunosuppressant drug administered to the patient, the first time, or a combination thereof. Some methods may include calculating the first immunosuppressant ratio, calculating the second dosage of the immunosuppressant drug based on the first immunosuppressant ratio, or a combination thereof. Additionally, or alternatively, the present methods may include obtaining a target range associated with a target serum level. In some such implementations, the second dosage of the immunosuppressant drug may be calculated based on the target serum level.

In some implementations, a method includes receiving an indication of a first dosage of an immunosuppressant drug given to a patient on a first day, receiving an indication of a first serum level of the patient associated with the first day, and generating a first output associated with a second dosage of the immunosuppressant drug calculated based on a first immunosuppressant ratio between the first dosage and the first serum level. Some such methods may include receiving a plurality of indications of a first dosage (e.g., at different times during the first day), receiving a plurality of indications of a first serum level, or both. The first serum level may be an amount of the immunosuppressant drug in the blood stream of the patient after receiving the first dosage. In some such implementations, the second dosage is associated with a second day that immediately follows the first day. For example, the first dosage may be administered on Monday and the second dosage may be administered on Tuesday. Thus, some of the foregoing methods allow for calculation of an immunosuppressing drug dosage using data acquired during a time period that is less than or equal to three days, such as data acquired during a time period that is less than or equal to twenty-four hours (e.g., a single day). The present systems and methods may therefore be utilized within the first few days following surgery and before one or more physiological conditions of the patient is stabilized.

Some aspects of the present methods also may include determining a target range associated with a target serum level. Additionally, calculating the second dosage of the immunosuppressant drug may be based on the target serum level. In some methods, the first immunosuppressant ratio is a ratio of the first dosage to the first serum level. In some such methods, calculation of the second dosage includes multiplying the first immunosuppressant ratio by the target serum level. For example, the calculation may be independent of a measured AST level, an ALT level, a GFR level, or a combination thereof. In some of the present methods, the first day is a twenty-four hour period that occurs within five days immediately following an organ transplant operation. In some methods, the first day is a twenty-four hour period within three days immediately following an organ transplant operation.

Some of the present methods may include determining a second serum level of the patient associated with the second day, calculating a third dosage of the immunosuppressant drug based on a second immunosuppressant ratio between the second dosage and the second serum level, and generating a second output based on the third dosage, the third dosage associated with a third day. The second serum level may be an amount of the immunosuppressant drug in the blood stream of the patient after receiving the second dosage. In some implementations, the second day immediately precedes the third day. Some methods may further include receiving an indication of a first aspartate transaminase (AST) level of the patient associated with the first day, receiving an indication of a second AST level of the patient associated with the second day, receiving an indication of a third AST level of the patient associated with the third day, or a combination thereof. One or more serum coefficients may be calculated based on one or more AST levels. For example, the serum coefficients may be calculated based on each of the AST levels being within a threshold range, such as, being within 10% of one another or steadily increasing or decreasing for multiple, consecutive days. Some such methods further include calculating a fourth dosage of the immunosuppressant drug based on a ratio between the third dosage and a third serum level, the first AST level, the second AST level, and the serum coefficients.

Another aspect of the present disclosure describes systems for calculating dosages for variable dosing medications. In some such aspects, the system may include a memory storing one or more instructions and a processor coupled to the memory and configured to execute the one or more instructions to perform one or more of the methods described herein. For example, the processor may be configured to execute the one or more instructions to determine a first dosage of an immunosuppressant drug given to a patient on a first day, determine a first serum level of the patient associated with the first day, calculate a second dosage of the immunosuppressant drug based on a first ratio of the first dosage to the first serum level, and initiate a first output associated with the second dosage.

In some implementations, the processor is configured to receive an input associated with a target serum level and calculate the second dosage by multiplying the first ratio by the target serum level. In some aspects, the processor of the present system is configured to store the first ratio, store the second dosage, determine a second serum level of the patient associated with a second day, and calculate a third dosage of the immunosuppressant drug based on a second ratio of the second dosage to the second serum level. The first day may immediately precede the second day. In some such systems, the processor is configured to initiate a second output associated with the third dosage. In some implementations, the system may include a display device and the first or second output is displayed on the display device. In some implementations of the system, determining the first serum level includes receiving, from a medical testing device, medical data associated with the patient on the first day and identifying the first serum level from the medical data.

In some implementations of the present systems, the processor may be configured to determine, select, or identify one or more patient biological factors. For example, the process may be configured to determine a first AST or ALT level of the patient associated with the first day, determine a second AST or ALT level of the patient associated with the second day, and determine a third AST or ALT level of the patient associated with a third day. In some such implementations, based on each of the AST or ALT levels being within a threshold range, the processor may calculate serum coefficients. To illustrate, the processor may compare the AST level or the ALT level to the threshold range and may calculate serum coefficients based on a determination that the AST level or the ALT level to the threshold range. In such implementation, the processor can determine a third serum level of the patient associated with the third day and calculate a fourth dosage of the immunosuppressant drug based on a ratio of the third dosage to the third serum level, the third AST or ALT level, and the serum coefficients.

In another aspect of the present disclosure a method for treating a patient after an organ transplant includes determining an immunosuppressant ratio based on a first dosage of an immunosuppressant drug administered to a patient at a first time and a first serum level of the patient associated with the first time. For example, the first serum level may be determined based on a blood sample taken within 24 hours of the first time. Some methods include administering a second dosage of the immunosuppressant drug to the patient during a second time period. The second dosage of the immunosuppressant drug may be determined based on the immunosuppressant ratio. The first time period may immediately precede the second time period. In some implementations, the method includes calculating the second dosage of the immunosuppressant drug based on the immunosuppressant ratio.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The terms “a” and “an” are defined as one or more unless this disclosure explicitly requires otherwise.

The term “about” as used herein can allow for a degree of variability in a value or range, for example, within 10%, within 5%, or within 1% of a stated value or of a stated limit of a range, and includes the exact stated value or range. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed implementation, the term “substantially” may be substituted with “within [a percentage] of” what is specified, where the percentage includes 0.1, 1, or 5 percent; and the term “approximately” may be substituted with “within 10 percent of” what is specified. The statement “substantially X to Y” has the same meaning as “substantially X to substantially Y,” unless indicated otherwise. Likewise, the statement “substantially X, Y, or substantially Z” has the same meaning as “substantially X, substantially Y, or substantially Z,” unless indicated otherwise. Unless stated otherwise, the word or as used herein is an inclusive or and is interchangeable with “and/or.” To illustrate, A, B, or C includes: A alone, B alone, C alone, a combination of A and B, a combination of A and C, a combination of B and C, or a combination of A, B, and C. Similarly, the phrase “A, B, C, or a combination thereof” or “A, B, C, or any combination thereof” includes: A alone, B alone, C alone, a combination of A and B, a combination of A and C, a combination of B and C, or a combination of A, B, and C.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any implementation of any of the systems, methods, and article of manufacture can consist of or consist essentially of-rather than comprise/have/include-any of the described steps, elements, or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, the term “wherein” may be used interchangeably with “where”.

Further, a device or system that is configured in a certain way is configured in at least that way, but it can also be configured in other ways than those specifically described. The feature or features of one implementation may be applied to other implementations, even though not described or illustrated, unless expressly prohibited by this disclosure or the nature of the implementations.

Some details associated with the implementations are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the implementation in the view.

FIG. 1 illustrates a block diagram of a system for use in determining an immunosuppressant dosage.

FIG. 2 is a flowchart illustrating an example of a method of determining an immunosuppressant dosage for a patient after an organ transplant.

FIG. 3 is a flowchart illustrating an example of a method of for treating a patient after an organ transplant.

FIGS. 4A-4C are graphs showing a tacrolimus serum level, an immunosuppressant ratio, and an administered dosage of a first control patient in a clinical trial.

FIGS. 5A-5C are graphs showing a tacrolimus serum level, an immunosuppressant ratio, and an administered dosage of a first phenotypic personalized medicine (PPM) patient in the clinical trial.

FIGS. 6A and 6B are graphs showing a tacrolimus serum level and an immunosuppressant ratio of a second control patient in the clinical trial.

FIG. 7 is a graph showing the post-transplant length-of-stay (LOS) in the hospital duration of the hospital stay for the overall study population, the Control group, and the PPM group in the clinical trial

FIGS. 8A and 8B are graphs showing a percentage of days a subject suffered a large deviation in tacrolimus serum level for the entire hospital stay and for the first ten days post-transplant, respectively.

FIG. 8C is a graph showing the total number of days a subject suffered a large deviation in tacrolimus serum level for the Control and PPM groups.

FIGS. 9A and 9B are graphs showing a percentage of days a subject had a tacrolimus serum level outside the target range for the entire hospital stay and for the first ten days post-transplant, respectively.

FIG. 9C is a graph showing the total number of days a subject had a tacrolimus serum level outside the target range for the Control and PPM groups.

FIGS. 10A and 10B are graphs showing a mean area-under-the-curve (AUC) of the subjects for the entire hospital stay and for the first ten days post-transplant, respectively.

FIG. 10C is a graph showing the total AUC outside-of-target for the Control and PPM groups

FIG. 11A is a graph showing the number of days it took for the aspartate aminotransferase (AST) of a subject to normalize after the transplant procedure.

FIG. 11B is a graph comparing the number of days for AST normalization and the LOS in the hospital for the subjects of the PPM group.

FIGS. 12A and 12B are graphs showing a mean tacrolimus serum level of the subjects for the entire hospital stay and for the first ten days post-transplant, respectively.

FIG. 13 is a graph comparing a percentage of days a subject had a tacrolimus serum level outside the target range and the LOS in the hospital for the subjects of the Control group.

### DETAILED DESCRIPTION

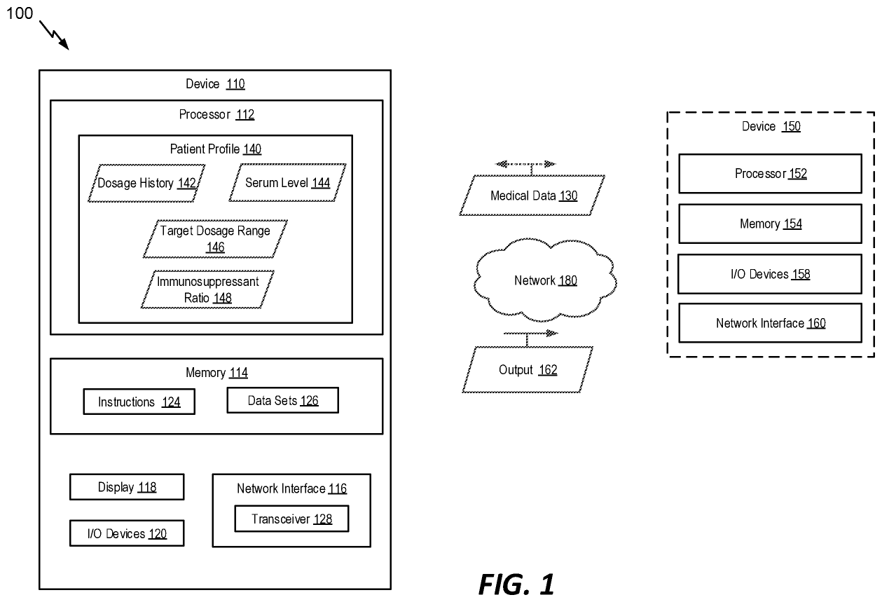

FIG. 1 is a block diagram of an example of a system 100 (PPM system) for use in determining an immunosuppressant dosage. System 100 may be used to monitor a concentration of a chemical substance (e.g., medical drug) within a patient's blood stream, determine whether to maintain or modify a drug dosage, determine a target drug dosage, or a combination thereof. As described herein, system 100 may be utilized for calculation of one or more dosages for a medication having variable dosing.

System 100, such as a variable dosing system, includes a first device 110 having a processor 112 and a memory 114. First device 110 may include or correspond to an electronic device, such as a smartphone, a tablet computing device, a personal computing device, a laptop computing device, a desktop computing device, medical device, a computer system, a cloud computing device, a server, or one or more distributed devices, as illustrative, non-limiting examples. In some implementations, system 100 may, but need not, include a second device 150, such as a user device. In some implementations, second device 150 is a mobile device (e.g., a smartphone or tablet) storing an application and first device 110 is a server that hosts or otherwise supports the application. Although system 100 is shown as having a single first device (e.g., 110) and a single second device (e.g., 150), system 100 is not to be limited. For example, system 100 may include multiple second devices (e.g., 150), such as a first user device associated with a first person and a second user device associated with a second person. The first person may include a first patient or a first hospital work, such as a doctor or a nurse, and the second person may include a second patient or a second hospital worker.

First device 110 includes processor 112, memory 114, and a network interface 116. In some implementations, first device 110 may include one or more other components such as a display 118, one or more input/output (I/O) devices 120, or the like. Processor 112 may be coupled to memory 114, network interface 116, display 118, or one or more I/O devices 120. Processor 112 may be a general purpose computer system (e.g., a personal computer (PC), a server, or a tablet device), a central processing unit (CPU), a special purpose processor platform (e.g., application specific integrated circuit (ASIC) or system on a chip (SoC)), or other computing circuitry. Processor 112 may include one or more processors, such as a baseband processor, an application processor, a graphics processor, or a combination thereof, as illustrative, non-limiting examples.

Processor 112 is configured to obtain a target dosage 132, an immunosuppressant ratio 134, or a combination thereof. For example, processor 112 may be configured to perform one or more operations, calculations, or otherwise determine target dosage 132 or immunosuppressant ratio 134. In other implementations, processor 112 may be configured to receive, select, or otherwise obtain target dosage 132 or immunosuppressant ratio 134, as described herein.

Memory 114 includes instructions 124 and one or more data sets. As an illustrative, non-limiting example, at least one of the data sets includes or corresponds to a patient profile 140 that is associated with a patient, such as an organ transplant recipient. Patient profile 140 can include one or more items or variables, such as, for example, past or current measurements, thresholds, ranges, or calculated values, as illustrative, non-limiting examples. To further illustrate, patient profile 140 may include various medical information of the patient, such as a dosage history 142, a serum level history 144, a target dosage range 146, a combination thereof, or the like, as illustrative, non-limiting examples.

The data stored in patient profile 140 may be accessible to or accessed by processor 112. For example, processor 112 may obtain, update, or store data to one or more of dosage history 142, serum level history 144, or target dosage range 146. Additionally, or alternatively, processor 112 may select data from patient profile 140 (e.g., dosage history 142, serum level history 144, or target dosage range 146) to use in calculations or other operations performed by processor 112 or another device. For example, processor 112 may use data from patient profile 140 to determine target dosage 132 or immunosuppressant ratio 134. In some implementations, processor 112 may update patient profile 140, or data associated therewith, regularly, periodically (e.g., every few seconds, minutes or hours), or continuously and the data associated with each updated patient profile 140 can be utilized to determine a dosage. In this way, the functions (e.g., function 1 and 2, described below) and calculations described herein can utilize several data points to determine the dosage. Additionally, or alternatively, such updates allow processor 112 to determine the dosage using recent data.

Dosage history 142 may include information associated with one or more drugs administered to the patient. For example, dosage history 142 may include one or more previously administered dosages of a drug (e.g., an amount or time of an administered drug, amount of the drug administered on previous day(s)), a number of doses of the drug per day, time of or between doses of the drug (e.g., consecutive doses of the same drug), a time since first administration of the drug, information associated with a most recent dosage of the drug, a combination thereof, or the like, as illustrative, non-limiting examples. To illustrate, dosage history 142 may include a first dosage 143 administered to the patient at a first time, such as a first dosage time. First dosage 143 can include dosage data, such as, for example, a dosage amount, a time associated with the start of the dosage being administered, a time associated with a completion of the dosage being administered, a time period during which the dosage was administered, a time dosage information was input/entered, a time associated with a sample (e.g., a blood sample) taken or to be taken after the dosage was administered, a drug name associate with the dosage, a drug batch number associated with the dosage, an identifier of a person administering the dosage, or a combination thereof. In some implementations, dosage data can include other information such as identification of the drug administrator (e.g., doctor, nurse, technician, etc.), a blood type of the patient, a white blood cell count of the patient, other biological markers associated with the patient, one or more biological markers associated with the transplanted organ, or the like, as illustrative, non-limiting examples. Although dosage history 142 is shown having only first dosage 143, the dosage history may include dosage data for each of a plurality of dosages administered to a patient associated with patient profile 140.

Serum level history 144 may include drug concentration (e.g., grams per liter of blood) of the one or more drugs administered to the patient at specific times or time periods. To illustrate, serum level history 144 can include a first serum level 145 associated with a first time, such as a serum level time. First serum level 145 may correspond to the drug concentration of the patient as a result of first dosage 143. For example, first serum level 145 may be the drug concentration of the patient at a time following the administration of first dosage 143 (e.g., 1, 2, 5, 10, 15, 20, 30, 45, 60 minutes or 1.5, 2, 3, 4, 5, 6, 7, 8, 9, 10, 11, 12, 14, 18, or 24 hours after administration of first dosage 143) or the average drug concentration for a time period following the administration of the first dosage, as non-limiting examples. Although serum level history 144 is shown having only first serum level 145, the serum level history may include serum level data corresponding to each of a plurality of dosages administered to a patient associated with patient profile 140.

Target dosage range 146 may include a target drug concentration for a patient. In some implementations, target dosage range 146 is equal to or between any two of the following: 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5 or 15.0 ng/mL, such as between 8.0 to 10.0 ng/mL. In some implementations, target dosage range 146 may be received via an input at first device 110 or stored within memory 114 and selected by processor 112. For example, memory 114 may include a table (or other data structure) and processor 112 may select the target dosage range based on the data stored within the memory. In some implementations, the selection may be presented to health care professional, such as a doctor, and the doctor provides an input to accept the selection or to modify the selection. Additionally, or alternatively, the processor 112 may provide (e.g., present) the patient data used to make the selection. The target dosage range may be determined based on one or more clinical factors or protocols associated with the patient, such as the patient's medical history, demographics, disease process, or the like.

In an illustrative, non-limiting example, dosage history 142 includes first dosage 143 of an immunosuppressant drug (e.g., tacrolimus) given to the patient on a first day, serum level history 144 includes first serum level 145 that is a concentration of the immunosuppressant drug (e.g., nanograms of tacrolimus per milliliter of blood) associated with the first day, and target dosage range 146 may include the preferred or target drug concentration of the immunosuppressant drug for the patient.

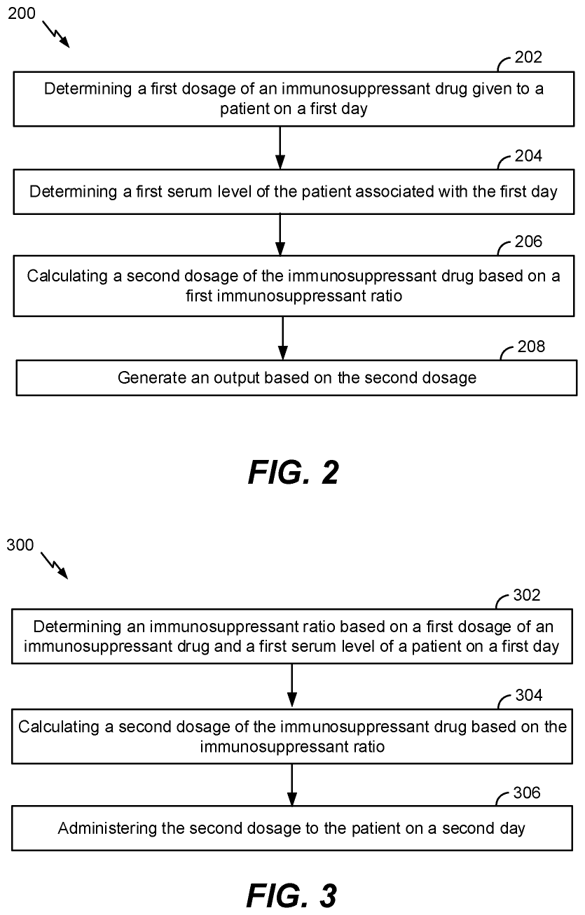

In some aspects, memory 114 may store the instructions 124 that, when executed by processor 112, cause the processor 112 to perform operations according to aspects of the present disclosure, as described herein, such as one or more operations as described with reference to at least FIGS. 2 and 3. Memory 114 may include read only memory (ROM) devices, random access memory (RAM) devices, one or more hard disk drives (HDDs), flash memory devices, solid state drives (SSDs), other devices configured to store data in a persistent or non-persistent state, or a combination of different memory devices.

Network interface 116 may be configured to communicatively couple first device 110 to one or more external devices, such as second device 150, via one or more networks 180. For example, network interface 116 includes a transceiver 128 configured to send and receive electronic signals. Although described as a transceiver, network interface 116 may alternatively include a receiver, a transmitter, or both. In some implementations, the network interfaces 116 includes a wireless interface such as a LoRa interface, a Wi-Fi interface (e.g., an IEEE 802.11 interface), a cellular interface, a Bluetooth interface, a BLE interface, a Zigbee interface, another type of low power network interface, or the like.

In some implementations, first device 110 includes display 118, one or more I/O devices 120, or a combination thereof. Display 118 can include any suitable device configured to present visual information. For example, display 118 may include a liquid crystal display (LCD), light-emitting diode (LED) display, electroluminescent (ELD) display, plasma display, a touchscreen display, or the like. The one or more I/O devices 120 may include one or more switches, one or more display devices, a stylus, a mouse, a keyboard, one or more touchscreens, a microphone, a camera, one or more speakers, one or more light sources, or other types of components that enable a user to receive information from or provide information to first device 110.

In some implementations, first device 110 may be a single device configured to perform the operations described herein. Although FIG. 1 shows first device 110 as a single block, the implementation of first device 110 is not limited to a single component, and instead may be distributed over several components. For example, operations of first device 110 may be distributed over multiple devices configured to perform all or a portion of the operations of first device 110 in accordance with the present disclosure. Implementing first device 110 functionality over multiple devices may increase efficiency, processing time, and reliability of system 100.

First device 110 is configured to receive or transmit medical data 130. For example, medical data 130 is received by first device 110 (e.g., via a user input, second device 150, network 180, other external device) and is stored in memory 114 and provided to processor 112. In other implementations, medical data 130 may be input into first device 110 in any other suitable manner. Medical data 130 may include information or measurements of a patient such as, for example, the patient's respiratory rate, heart rate, diastolic blood pressure, blood toxicology, toxicity, blood cell count, liver functions (e.g., AST data, ALT data, or the like), kidney functions (e.g., GFR data), age, quantitative/non-subjective mental status, pulse pressure, height, weight, biological sex, site of transplant (kidney, liver, etc.), date of transplant operation, or other clinical or demographical information of the patient. In some implementations, based on medical data 130, processor 112 can identify, calculate, sort, or otherwise determine one or more patient profiles 140 or data associated with the one or more patient profiles. Processor 112 may then utilize the information of patient profile 140 and provide an output, such as output 162. The data within dosage history 142, serum level history 144, or target dosage range 146 may be included in medical data 130 or, alternatively, processor 112 may calculate at least a portion of the data within dosage history 142, serum level history 144, or target dosage range 146. In some implementations, medical data 130 may be updated (e.g., via instructions sent from first device 110) at selected or predetermined intervals, such as, for example, every 5, 10, 15, 20, 30, 40, 50 or 60 seconds or every 2, 3, 5, 10, 15, 20, 30, 40, 50, or 60 minutes, or every 2, 4, 6, 8, 10, 12, 14, 16, 18, 20, or 24 hours. Processor 112 may evaluate (e.g., calculate or otherwise determine), one or more patient profiles 140 or data associated with the one or more patient profiles each time medical data 130 is updated. In this way, and others, first device 110 may continuously update data and utilize the data to more accurately predict a next dosage.

Some implementations of system 100 include a second device 150 configured to be in communication with first device 110. Second device 150 may be associated with a patient or a health care provider (such as a nurse, a physician, or the like) in a hospital ward, at the patient's home, or other places, to evaluate a patient's medical treatment. Second device 150 includes one or more processors 152, a memory 154, one or more input/output (I/O) devices 158, and a network interface 160.

Processor 152 may include one or more forms of processor-based systems in accordance with aspects described herein. For example, processor 152 may include a general purpose computer system (e.g., a personal computer (PC), a server, a tablet device, etc.) or a special purpose processor platform (e.g., application specific integrated circuit (ASIC), system on a chip (SoC), etc.). Memory 154 may include ROM devices, RAM devices, one or more HDDs, flash memory devices, SSDs, other devices configured to store data in a persistent or non-persistent state, or a combination of different memory devices. In aspects, memory 154 may store the instructions that, when executed by processor 152, cause the processor 152 to perform operations. The instructions may be in the form of software, or software applications downloadable to second device 150.

The one or more I/O devices 158 may include a display device, a touchscreen, a button, a switch, a camera, a mouse, a keyboard, other I/O devices, or a combination thereof. The network interface 160 may be configured to communicatively couple the second device 150 to one or more external devices, such as first device 110.

In some implementations, second device 150 receives patient data, such as medical data 130, which may be stored at memory 154. The patient data may be associated with or correspond to one or more patients. The patient data (e.g., 130) may include information or measurements of a patient, such as a list of one or more drugs used in the patient's treatment, concentrations of the one or more drugs in the patient's system, and previous or future dosages of the one or more drugs. In an illustrative example, the patient data may include a dosage of an immunosuppressant drug to be administered to the one or more patients on a specific day.

Network 180 may include may be configured to be communicatively coupled to one or more devices, such as first device 110 and second device 150. In some implementations, network 180 may include a server such as a general purpose processor-based system (e.g., PC or other server system having a processor, memory, suitable I/O functionality, and OS) operating under control of an instruction set to interface with first device 110. Network 180 may include a wired network, a wireless network, or a combination thereof. For example, network 180 may enable wired communication, wireless communication, or both, between first device 110 and second device 150. In wired implementations, I/O devices 120, 158 of first and second device 110, 150 may include a port, such as an Ethernet port or other wired connection port.

During operation, system 100 may obtain an indication of first dosage 143 associated with a first time. The indication of the first dosage 143 may include dosage data, as described above, or other information associated with a dose of a medication (e.g., immunosuppressant drug) administered to a patient at or during the first time. The first time can include a single time at which the drug is administered (e.g., for a single injection, ingestion of a pill, or other single use administration) or a period of time over which the drug is administered (e.g., intravenous drip).

In some implementations, system 100 may be configured to obtain an indication of a first patient sample, such a blood sample. Additionally, or alternatively, system 100 may be configured to obtain an indication of a result associated with the first patient sample. The result of the first sample can include first serum level 145, respiratory rate, heart rate, diastolic blood pressure, blood toxicology, toxicity, blood cell count, liver functions (e.g., AST data, ALT data, or the like), kidney functions (e.g., GFR data), pulse pressure, or other clinical or demographical information of the patient. In some implementations, system 100 may be configured to measure the first patient sample or analyze the first patient sample to obtain the result of the first sample. To illustrate, system 100 may include equipment configured to record measurements of the first patient sample. Additionally, or alternatively, system 100 may be in communication with such equipment and can receive (e.g., as a result of a request sent by processor 112) the measurements or other information associated with the first patient sample. In other implementations, the measurements or other information associated with the first patient sample may be input into system 100 or otherwise obtained by system 100, such as received in medical data 130.

First dosage 143, the first patient sample, or the result of the first patient sample may occur during or be associated with a first time period. First time period can correspond to a four hour time period, a six hour time period, a twelve hour time period (e.g., twice a day), an eighteen hour time period, a twenty-four hour period (single day), during a thirty-six hour time period, during a forty-eight hour time period, during a sixty hour time period, a seventy-two hour time period, or any other time period, such as a time period that is less than or equal to a five day period. In such implementations, the first time period includes the first time. For example, first dosage 143 may be administered at the first time and, at a later time within the first time period, the first patient sample may be taken and results of the first patient sample obtained. As a further example, first patient sample may be taken, or results of the first patient sample obtained, at a time within the first time period that is at least 1, 5, 10, 15, 20, 30, 40, 45 or 50 minutes after the first time, or 1, 2, 3, 4, 5, 6, 7, 8, 9, 10, 11, 12, 14, 16, 18, 24, or 36 hours after the first time.

In some aspects, system 100 is configured to determine a second dosage (e.g., target dosage 132) associated with a second time. Second dosage may be determined based on the first patient sample or the results of the first patient sample, such as first serum level 145. The second dosage can include dosage data, as described above, or other information associated with a dose of a medication (e.g., immunosuppressant drug) to be administered to a patient during the second time. The second time is subsequent to the first time and, in some implementations, the second time is included within a second time period. The second time period can have a duration that is the same, or different than, the duration of the first time period and at least a portion of the second time period is after the first time. In some implementations, the second time period is distinct from the first time period, while in other implementations, the second time period may partially overlap the first time period. System 100 may determine the second dosage during the first time period (e.g., after receiving the result of the first patient sample) or during the second time period (e.g., before the second time). System 100 may generate an output (e.g., 162) of the second dosage and may present the output via a display or transmit the output to another device.

System 100 may obtain an indication of a second patient sample or an indication of a result associated with the second patient sample. The result of the second sample can include a second serum level associated with the second time period. In some implementations, second dosage, the second patient sample, or the result of the second patient sample may be obtained during the second time period. For example, the second dosage may be administered at the second time and, at a later time within the second time period, the second patient sample may be taken and results of the second patient sample obtained.

In some aspects, system 100 is configured to determine a third dosage (e.g., target dosage 132) associated with a third time that is after the second time. In some such implementations, the third dosage may be determined based on the result of the second patient sample, such as the second serum level. In some implementations, system 100, via processor 112, may be configured to store target dosage 132 within dosage history 142 as a previous dosage after the target dosage is calculated or otherwise determined. System may then use the previous dosage to determine a next target dosage (e.g., 132).

In some aspects, system 100 may be configured to determine a daily target dosage (e.g., 132) for an immunosuppressant drug that is prescribed to a patient, as described below. Although the illustrative examples described herein refer to determining a dosage for a particular day (e.g., calendar day), system 100 may also operate to determine dosages for any time period such as a six hour time period, a twelve hour time period (e.g., twice a day), an eighteen hour time period, a twenty-four hour period (single day), during a thirty-six hour time period, during a forty-eight hour time period, during a sixty hour time period, a seventy-two hour time period, or any other time period, such as a time period that is less than or equal to a five day period. For example, in some implementations, system 100 may determine target dosage 132 of an immunosuppressant that is given twice daily, such as a dosage administered every twelve hours. In such implementations, target dosage 132 may be determined using data (e.g., first dosage 143 or first serum level 145) acquired during a time period that is less than or equal to twelve hours.

In daily dosage operations, processor 112 may determine, or otherwise obtain, a first dosage of an immunosuppressant drug (e.g., 143) given to a patient on a first day and a first serum level (e.g., 145) of the patient associate with the first day. For example, processor 112 may obtain an indication of a first serum level 145 of the patient associated with a first dosage 143 of an immunosuppressant drug administered to a patient on a first day, such as a twenty-four hour period or a calendar day (e.g., Monday, Tuesday, etc.). In some implementations, first serum level 145 corresponds to a drug concentration of the immunosuppressant drug resulting from first dosage 143.

Based on first dosage 143 or first serum level 145 of the patient, processor 112 may determine an immunosuppressant ratio 134 (e.g., first immunosuppressant ratio). Processor 112 may utilize immunosuppressant ratio 134 to calculate a second dosage (e.g., 132) of the immunosuppressant drug to be administered to the patient on a second day. The calculation of second dosage may occur during the first day or during the second day. In some implementations, processor 112 may generate an output (e.g., 160) that includes or corresponds to the second dosage. The second dosage may be associated with a second day that is different from the first day. In some implementations, the first day may immediately precede the second day. To illustrate, the first day may be Wednesday and the second day may be Thursday.

In some implementations, system 100 may determine additional dosages for one or more subsequent days. For example, processor 112 may obtain an indication of a second serum level of the patient associated with the second day. In such implementations, processor 112 may then determine a second immunosuppressant ratio (e.g., 134) based on the second dosage and the second serum level of the patient. In some such implementations, immunosuppressant ratio 134 is based on the first dosage (e.g., 143) to the first serum level (e.g., 145), such as, for example, the ratio of the first dosage to the first serum level. Second dosage can correspond to the previously determined target dosage (e.g., 132) associated with the second day. Processor 112 may utilize the second immunosuppressant ratio (e.g., 134) to calculate a third dosage (e.g., 132) of the immunosuppressant drug to be administered to the patient on a third day. In some implementations, the second day may immediately precede the third day. In this way and others, system 100 is configured to calculate an immunosuppressant dosage using only data from only a single, most recent time period such as a twenty-four hour period (e.g., previous day's medical data). System 100 can be employed immediately after transplant to manage immunosuppressant dosage and avoid or prevent under and overdosing immediately following a transplant operation.

In some implementations, processor 112 may be in communication with an external device to receive medical data (e.g., 130) or updates to the medical data that includes at least a portion of patient's dosage history 142 and serum level history 144. In one example, processor 112 may update or receive medical data at specific time intervals (e.g., every minute, every hour, every day, or any interval in between). Processor 112 may select a dosage (e.g., first dosage 143) from dosage history 142 that corresponds to an immunosuppressant dosage from a specific day and select a serum level (e.g., first serum level 145) from serum level history 144 that corresponds to the immunosuppressant serum level from the specific day. Processor 112 may then determine the immunosuppressant ratio 134 associated with the specific day and calculate target dosage 132 to be administered at a next day to maintain the patient's serum level within target dosage range 146. For example, the drug dosage may be calculated using the function:

$${D}_{T} = \frac{{D}_{T - 1} * {S}_{P}}{{S}_{T - 1}} \qquad ( 1 )$$

where D_{T} is the drug dosage to be administered to a patient, D_{T-1} is the drug dosage administered to the patient on the preceding day, S_{P} is a preferred or target serum level, and S_{T-1} is the patient's serum level on the preceding day.

In this way, system 100 may calculate a drug dosage independent from patient markers (e.g., AST level, ALT level, or GFR level), biological identifiers, or other clinical factors that may be unstable immediately following a transplant. Surprisingly, as described further herein with reference to the Examples, calculation of the drug dosage without utilizing patient-specific data provides a more accurate dosage than conventional methods that are specifically tailored to include patient-specific data, such as AST level, ALT level, or GFR level.

In some implementations, target dosage 132 may be calculated based on medical data (e.g., 130) from multiple days. To illustrate, when a patient's bodily markers (e.g., AST, ALT, GFR, etc.) begin to stabilize, processor 112 may be configured to use a different calculation to determine target dosage 132. In a non-limiting example, target dosage 132 may be calculated based on a patient's AST or ALT levels over multiple days once the patient is considered stable. For example, the drug dosage determined for a twenty-four hour period may be calculated using the function:

$${D}_{T} = {S}_{P} * ( {x}_{0} ( t ) + {x}_{1} ( t ) * {AST}_{T - 1} * {( \frac{D}{S} )}_{T - 1} + {z}_{11} ( t ) * {AST}_{T - 2} * {( \frac{D}{S} )}_{T - 2} ) \qquad ( 2 )$$

where D_{T}=the drug dosage to be administered to a patient on a fourth day; x₀(t)x₁(t), and z₁₁(t) are patient specific coefficients that are derived using a calibration test; AST_{T-1}=AST level of the patient on a second day; (D/S)_{T-1}=the immunosuppressant ratio (e.g., 134) of the second day; AST_{T-2}=AST level of the patient on a first day; and (D/S)_{T-2}=the immunosuppressant ratio (e.g., 134) of the first day. In some implementations, the values x₀(t)x₁(t), and z₁₁(t) change when calculating a drug dosage for any day subsequent to the fourth day. For example, when determining a drug dosage for a subsequent day, x₀(t)x₁(t), and z₁₁(t) may be different than when determining the drug dosage for the fourth day. In some implementations, the patient specific coefficients are associated with the three days immediately preceding the day for which the drug dosage is to be calculated. Some such patient specific coefficients are determined based on AST, ALT, GFR, an immunosuppressant ratio, or combination thereof. In some aspects, system 100 (e.g., processor 112) may receive or otherwise determine updated medical data (e.g., 130) during patient treatment. In some such implementations, the above described functions may utilize the updated data to generate dosages at a higher accuracy.

In some aspects of system 100, processor 112 may be configured to select between a first calculation and a second calculation. In some implementations, the selection can be based on a comparison of data to a threshold, such as a stability threshold. In an illustrative, non-limiting example, processor 112 may compare biological markers to the stability threshold and when the threshold is satisfied, the processor may select a first calculation (e.g., function (2)) associated with a stable patient calculation. Alternatively, when the threshold is not satisfied, the processor may select a second calculation associated with an unstable patient calculation (e.g., function (1)). In some implementations, the stability threshold may be based on AST, ALT, GFR, dialysis, other anti-infection drugs, or the like. In implementations when the biological markers correspond to the patient's AST, ALT and or eGFR levels, the processor 112 may compare AST or ALT levels for one or more recent time periods to a current AST or ALT level, respectively. Based on the recent time period's biological markers being within a stability range (e.g., such as within 2, 3, 5, 10, 15, 20, or 25% of the current marker), or based on the current marker continuing a trend (e.g., increasing for multiple consecutive days or decreasing for multiple consecutive days), processor 112 may determine the marker is stable. Based on the determination that a marker is stable, processor 112 may calculate target dosage 132 using data (e.g., 130) associated with multiple days (e.g., function (2)). In this way, and others, system 100 may determine a drug dosage that maintains the serum level within target dosage range 146 with more accuracy than conventional system allowing patients to stabilize quicker and decreasing time spent in the hospital.

In some implementations of system 100, processor 112 may generate an output 162 that provides an indication of the calculated drug dosage (e.g., target dosage 132). For example, output 162 may be transmitted to second device 150 to notify an individual (e.g., medical professional) of target dosage 132 for a current day or next day. Although output 162 is illustrated as being transmitted to second device 150, in other implementation, output may be transmitted to another external device or output via first device 110 (e.g., output from processor 112 to display 118 to depict information associated with the output.)

Output 162 may include or be provided with additional information, such as, for example, a time to administer the dose, a time period during which the dose is to be administered, an amount of the dose, a name of the drug, a time to acquire a sample for a serum level after administering the dose, a location for the dose to be administered, a responsible physician, or the like. In some implementations, output 162 may be associated with a change in drug dosage. In such implementations, processor 112 may compare a first dosage (e.g., previously calculated dosage) and a second dosage (e.g., target dosage 132) to determine if the dosage has changed. For example, processor 112 may calculate a difference between a first dosage and a second dosage. The difference may then be compared to a dosage threshold (e.g., difference in dosage less than or equal to 0.1, 0.25, 0.5, 1.0, 1.5, 2.0, 2.5, 3.0, 3.5, 4.0 or 5.0 ng). Based on the difference being greater than or equal to the dosage threshold, processor 112 may transmit output 162 that initiates an alert at first device 110, second device 150, or both. Accordingly, system 100 may notify a physician or patient of a change in drug dosage from the previously administered dosage.

In some implementations, output 162 may correspond to a drug dosage calculated via function (1), (2), or both. For example, processor 112 may compare one or more patient markers (e.g., AST, ALT, GFR) with a previous day's patient marker and, based on the comparison exceeding a predetermined threshold, calculate a drug dosage using function (1) and output the drug dosage. Additionally, or alternatively, based on the comparison being below the predetermined threshold, processor 112 may calculate a drug dosage using function (2) and output the drug dosage. Output 162 may also include one or more patient markers such that a medical professional may determine which of determined dosages should be given to the patient. For example, output 162 may include serum level, a first dosage calculated using data from a single day, a second dose calculated using data from multiple days, AST levels, ALT levels, other stability information, a recommendation between the first and second dosage, other information, or a combination thereof.

Upon receiving output 162 (e.g., at second device 150 or at display 118), a representation of output 162 may be displayed or certain action(s) of treatment for the patient may be triggered, such as automatically notifying a medical professional. In some implementations, second device 150 may be configured to selectively notify an operator via an alert. For example, based on an output (e.g., 160) corresponding to a drug dosage that is different than a previous days drug dosage, processor 152 may initiate an alert to notify a medical professional that the daily dosage has changed. In some implementations, second device 150 may be configured to request output 162 from first device 110. As an illustrative, non-limiting example, a medical professional be assigned a second device 150 and request an output (e.g., 160) associated with a particular patient profile (e.g., 140). In such implementations, the medical professional may request the drug dosage associated with the patient profile and first device 110 may transmit the drug dosage to second device 150. In implementations where system 100 includes multiple second devices, such as several user devices, a first user device corresponding to a lab can provide medical data 130 to first device 110 and the first device can provide output 162 to the first user device, to a second user device corresponding to a physician, or both.

Although the above examples of system 100 are described with respect to a calendar day, in other implementations, system 100 may be configured to calculate dosages (e.g., 132) associated with a different time period such as such as a six hour time period, a twelve hour time period, an eighteen hour time period, a twenty-four hour period (other than a calendar day), during a thirty-six hour time period, during a forty-eight hour time period, during a sixty hour time period, a seventy-two hour time period, or any other time period less than or equal to a five day period. In such implementations, processor 112 may operate based on the respective time period instead of specific days. Likewise, although described as operating for a single patient, system 100 may be configured to calculate, for each of multiple patients, a drug dosage. For example, processor 112 may receive or store a plurality of patient profiles 140, each of which are associated with specific patient. In some such implementations, processor 112 may analyze medical data 130 and select a dosage history (e.g., 142), serum level history (e.g., 144), target dosage range (e.g., 146), or a combination thereof, for each patient. As a non-limiting example, medical data 130 may include patient identifiers (IDs) that correspond to a specific patient profile (e.g., 140).

In some implementations, first device 110 executes an algorithm that includes calculations of patient history data (e.g., 130) including determining slopes, functions, and other relationships between the patient history data. The algorithm may be a learning algorithm and will continue machine learning of all uploaded/received data so that the algorithm becomes more accurate as the database (e.g., stored patient data) grows—e.g., increases in data received. For example, the algorithm will continue machine learning of all uploaded data (e.g., training data) so that it gets more accurate in dose calculation as it grows.

In some implementations, system 100 is configured to obtain an indication of a first serum level (e.g., 145) of the patient associated with a first dosage (e.g., 143) of an immunosuppressant drug administered to a patient at a first time. System 100 is further configured to generate a first output (e.g., 162) that indicates a second dosage (e.g., 132) of the immunosuppressant drug calculated based on a first immunosuppressant ratio (e.g., 134). The first immunosuppressant ratio may be based on the first dosage and the first serum level. Additionally, the second dosage is associated with a second time that is subsequent to the first time.

In some implementations, system 100 is configured to determine immunosuppressant ratio 134 based on a first dosage of an immunosuppressant drug (e.g., 143) and a first serum level (e.g., 145) of a patient associated with a first time period. System 100 is further configured to output an indication of a second dosage (e.g., 132) of the immunosuppressant drug to be administered to the patient on a second time period. The second dosage (e.g., 132) of the immunosuppressant drug may be determined based on the immunosuppressant ratio 134. The first time period may precede the second time period. In some implementations, the system 100 is further configured to calculate the second dosage (e.g., 132) of the immunosuppressant drug based on immunosuppressant ratio 134.

Accordingly, system 100 advantageously provides a computational and software platform for determining a drug dosage for a patient, specifically for determining an immunosuppressant dosage (e.g., tacrolimus dosage) using data from a single time period. Additionally, or alternatively, the dosage may be determined independent of patient biological markers and can be utilized first few days after organ transplant. Surprisingly, despite using patient data from a single time period (e.g., single day), system 100 may have increased effectiveness as compared to traditional calculated dosages that required the previous three days patient data. Thus, a physician may provide more accurate dosing in the days immediately following a transplant procedure without having to wait for patient markers to stabilize while also allowing for changes in the medical regimen.

Referring to FIGS. 2 and 3, methods of determining a dosage are shown. For example, each of the methods of FIGS. 2 and 3 may be performed by system 100, such as first device 110 (e.g., processor 112), second device 150 (e.g., processor 152), or both.

Referring to FIG. 2, a method 200 of calculating an immunosuppressant drug dosage for a patient includes obtaining a first dosage of an immunosuppressant drug given at a first time associated with a first time period, at 202. For example, the first dosage may correspond to a tacrolimus dosage given to the patient and may be determined by selecting the first dosage from a plurality of dosages accessible from medical data (e.g., 130). In some implementations, first dosage may be determined by receiving an input (e.g., via I/O device 120, second device 150, or other external device). First time period may be a time period between doses of the immunosuppressant drug, such as a six hour time period, a twelve hour time period, an eighteen hour time period, a twenty-four hour period (calendar day), during a thirty-six hour time period, during a forty-eight hour time period, during a sixty hour time period, a seventy-two hour time period, or any other time period less than or equal to a five day period.

The method 200 also includes obtaining a first serum level of the patient associated with the first time period, at 204. First serum level may correspond to a tacrolimus drug concentration (ng/ml). Similar to that described with first dosage, the first serum level may be selected or calculated from medical data (e.g., 130) or received via an input. The first dosage and first serum level (or a sample to determine the first serum level) may be obtained at different times during the first time period. For example, a first dosage of the immunosuppressant drug may be administered to a patient and, after a suitable time has elapsed to allow the drug to be absorbed in the patient's blood stream, the first serum level may be obtained. In other implementations, first dosage and first serum level may be selected from a patient profile, or other data set, at approximately the same time (e.g., within thirty seconds). In some such implementations, first dosage and first serum level are recorded and can be identified at a later date.

The method 200 also includes determining a second dosage of the immunosuppressant drug based on a first immunosuppressant ratio, at 206. In some implementations, the first immunosuppressant ratio is calculated based on a ratio between the first dosage and the first serum level. For example, first immunosuppressant ratio may be a ratio of the first dosage to the first serum level. In some implementations, one or more additional factors may be utilized to calculate the second dosage. For example, a target drug concentration (e.g., target dosage range) may be determined and the second dosage calculated based on the first immunosuppressant ratio and the target drug concentration. As a non-limiting example, calculating the second dosage includes multiplying the first immunosuppressant ratio by the target serum level. In other implementations, one or more patient-specific coefficients or patient markers may be used to calculate the second dosage. The patient-specific coefficients may be experimentally determined using patient data from one or more time periods (e.g., by calibrating phenotypic outputs of a specific patient and the drug-dose inputs). The patient markers may include liver markers, kidney markers, other biomarkers, or the like.

The method 200 also includes generating a first output based on the second dosage, at 208. The first output may provide an indication of the second dosage. In some implementations, first output may cause a device (e.g., 110, 150) to display the second dosage. In this way a physician may identify the second dosage and properly administer or prepare the correct dosage for the patient. In other implementations, first output may indicate a change in the dosage (e.g., difference between first dosage and second dosage). The second dosage may be associated with a second time period that is subsequent to the first time period. In some implementations, the first time period may immediately precede the second time period (e.g., second time period includes the 24 hours immediately following the first time period).

Referring to FIG. 3, a method 300 of treating a patient after an organ transplant includes determining an immunosuppressant ratio based on a first dosage of an immunosuppressant drug and a first serum level of a patient on a first day, at 302. In some implementations, the immunosuppressant ratio is the ratio of the first dosage to the first serum level.

The method 300 also includes calculating a second dosage of the immunosuppressant drug based on the immunosuppressant ratio, at 304. In some implementations, the calculation includes multiplying the immunosuppressant ratio by a target serum level. In some such implementations, no other patient history data is included in the calculation. In other implementations, one or more additional patient factors may be included in the calculation such as, for example, a patient-specific coefficient, AST levels, ALT levels, GFR level, or the like.

In some implementations, the method 300 also includes administering the second dosage to the patient on a second day, at 306. In some implementations, administering may include filling or ordering a prescription of the drug at the second dosage. In other implantations, administering may include providing the second dosage of the drug to the patient. Although method 300 is described as occurring for a specific day, the method may also be utilized for another time period, such as a twelve hour time period or other time period that is less than three days.

As part of the present disclosure, specific examples are included below. The examples are for illustrative purposes only and are not intended to be limiting. Those of ordinary skill in the art will readily recognize parameters that can be changed or modified to yield essentially the same results.

### EXAMPLE

### Comparative Analysis of the Present Dosage Calculation System and Other Method

Several adults scheduled to undergo primary or redo liver or simultaneous liver and kidney transplantation were assessed for eligibility in a clinical trial in which calculation of a tacrolimus dosage was performed in an attempt to maintain the patient's tacrolimus trough blood levels (tacrolimus serum level) within a target range of 8-10 ng/mL. The inclusion criteria for the study was (1) the subject was undergoing primary or secondary liver or simultaneous liver and kidney transplantation; (2) the subject or a surrogate was able to provide informed consent; and (3) the subject was at least 18 years of age. Exclusion criteria included (1) enrollment in another investigational device or drug study; (2) prisoners or subjects who are compulsorily detained (involuntarily incarcerated); (3) subjects with any psychiatric or medical illness that, in the investigators' opinion, may put the subject at significant risk, may confound the study results, or may interfere significantly with the subject's participation in the study; and (4) patients with contraindications to tacrolimus or subjects that were pre-operatively anticipated to be switched to cyclosporine or a mammalian target of rapamycin (mTOR) inhibitor. Sixty two (62) subjects were accepted into the trial.

Each of the 62 selected subjects were randomized, with half (31) assigned to a control group and the other half (31) assigned to a phenotypic personalized medicine (PPM) group. The subjects were blinded to group assignments; the clinical team was not blinded. The patients in the PPM group were given tacrolimus dosages based on the present dosage calculation system and patients of the Control group were given tacrolimus dosages based on standard-of-care (SOC) dosing. The SOC dosing utilized a combination of dose titration and a subjective determination by a clinician. The subjective determination relied on patient weight, age, muscle mass, race/ethnicity, frailty, diet, concomitant medication, and other factors to select an initial dose for the next twenty-four hours. The patient's response to this dose is then taken into consideration for determining the next day's dose recommendation. This determination was repeated each day until the patient was able to be released from the hospital.

Two patients in the PPM group did not receive the intervention because their transplant operation was not performed after going to the operating room. Tacrolimus dosage was discontinued for one patient in each of the PPM group and the Control group. Both of these patients had neurotoxicity within the first week after transplant that was deemed not dose related, as evidenced by brain imaging. One patient assigned to PPM group did not have their tacrolimus level measured on post-operative day 5 due to a phlebotomy/laboratory error. As this would affect dosing accuracy and the study outcomes, this patient was excluded from analysis. One patient assigned to the Control group refused multiple tacrolimus doses in the post-operative period and was therefore excluded from analysis. The remaining 56 (29 Control group and 27 PPM group) patients completed the study and were discharged from the hospital after transplantation.

Table 1, reproduced below, shows a comparison of the clinical features (e.g., physical characteristics) of the overall population of the patients.

TABLE 1
Baseline characteristics of the study population

| | CONTROL GROUP | | PPM GROUP | |
|---|---|---|---|---|
| | (N = 29) | | (N = 27) | |
| Male | 16 | (55%) | 16 | (59%) |
| Female | 13 | (45%) | 11 | (41%) |
| HCC | 3 | (10%) | 7 | (26%) |
| SLKT | 5 | (17%) | 3 | (11%) |
| Redo OLT | 1 | (3%) | 1 | (4%) |
| Non-Hispanic White | 26 | (90%) | 23 | (85%) |
| Hispanic White | 1 | (3%) | 4 | (15%) |
| Non-Hispanic Black | 2 | (6%) | 0 | (0%) |
| Recipient age (years) | 57 | (41-61) | 58 | (51-64) |
| BMI (kg/m²) | 28.7 | (4.9) | 28.7 | (5.6) |
| NaMELD | 27 | (18.5-30.5) | 27 | (15-32) |
| Warm Ischemia Time (min) | 31 | (26.5-38.5) | 33 | (26-39) |
| Cold Ischemia Time (min) | 390 | (310-440) | 330 | (300-420) |
| DCD donor | 0 | (0%) | 1 | (4%) |
| Donor Age (years) | 41 | (25.5-58) | 38 | (32-54) |
| Hepatitis C Positive Donor | 1 | (3%) | 2 | (7%) |

HCC: hepatocellular carcinoma;
SLKT: simultaneous liver-kidney transplant;
OLT: orthotropic liver transplant;
BMI: body mass index;
NaMELD: sodium-model for end-stage liver disease

In the overall population of 56 patients, the median age was 58 (Q₁-Q₃ 48-62); 32 (57%) were male. The mean body-mass index (BMI) was 28.7 kg/m² (SD 5.2). The etiology for liver disease was alcohol related in 21 (37.5%), non-alcoholic steatohepatitis in 13 (23%), and hepatitis C in 10 (18%). Ten patients (18%) had hepatocellular carcinoma, 2 (4%) were undergoing a repeat liver transplantation, and 8 (14%) were undergoing a combined liver/kidney transplantation. As shown in Table 1, there were no significant differences in the baseline characteristics of the two groups.

Following liver transplantation, all study patients were started on standard-of-care medication regimen: a three-drug combination of tacrolimus, a corticosteroid, and mycophenolate. Blood tacrolimus trough levels were measured daily in the morning until patient discharge. For the first 3 days, each subject of both the PPM group and the Control group were given a dosage of tacrolimus according to the standard-of-care procedure, in which a clinical pharmacist, together with the transplant surgeon on call, determined the doses. After the third day, the Control group continued with the standard-of-care procedure while the PPM group subjects were given dosages calculated by the PPM prediction model, shown in the Function below.

$$c ( t + 1 ) = [ TSL ( t + 1 ) / TSL ( t ) ] * c ( t )$$

c(t+1) is the estimated next dosage; TSL(t+1) is the desired blood tacrolimus trough level; TSL(t) is the measured blood tacrolimus trough level; and c(t) is the previous dosage. The primary outcome measure was percent days with large (>2 ng/ml) deviations from target range. Secondary outcomes included percent days outside-of-target range, ratio of area-under-the-curve outside-of-target-range to total days, graft rejection, graft failure, death, infections, length of stay, nephrotoxicity episodes, or neurotoxicity episodes. Comparisons between the two groups were performed using either the two-tailed Welch's t-test or Student's t-test. For nonparametric (non-normal) distributions, a two-tailed Levene's test was used to compare variances and a two-tailed Wilcoxon Rank Sum test was used to compare medians. Logistic regression was used to dichotomize the length of stay after transplant and the percent days out of target range for comparison. Statistical analyses were performed on JMP Pro 16.1.0.

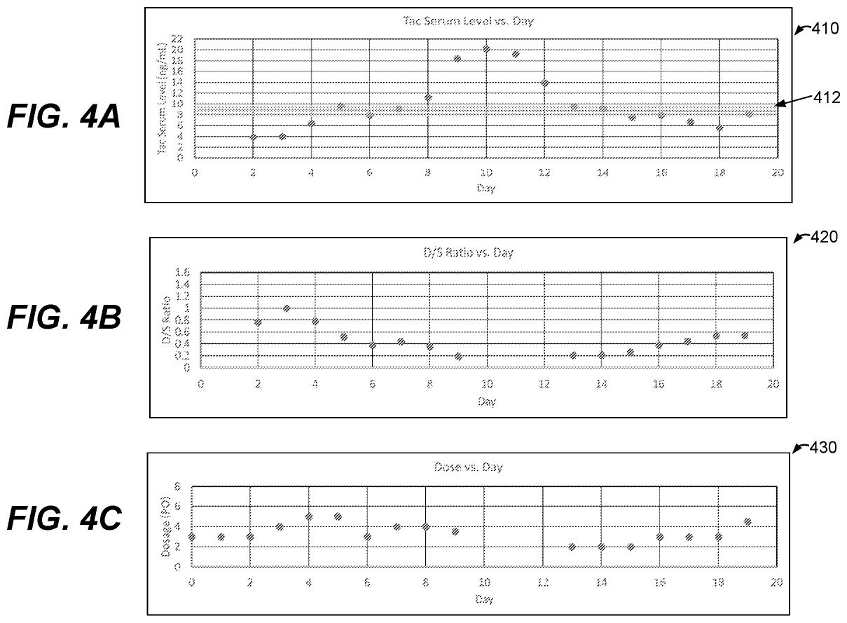

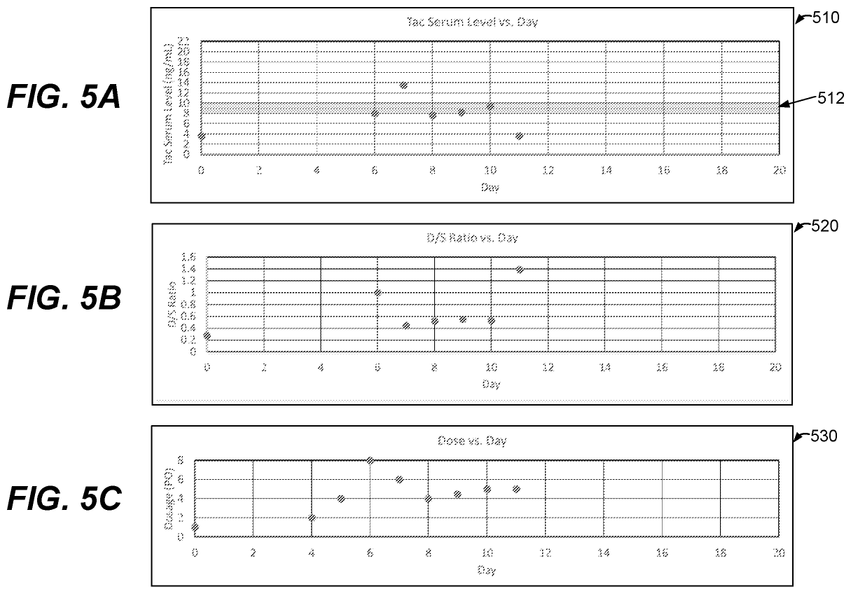

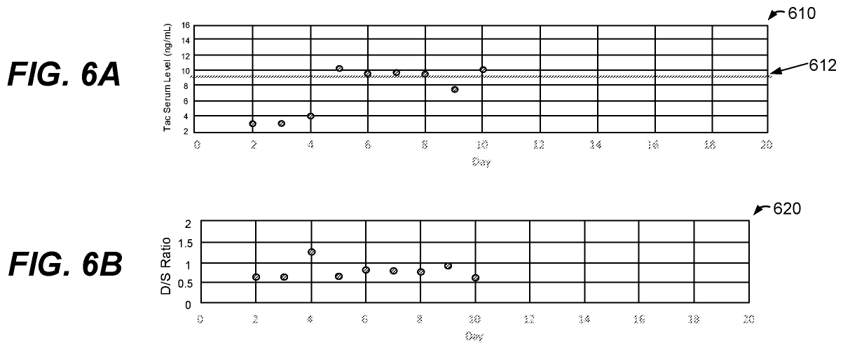

The difference in the dosing procedure for the Control group and the PPM group can be illustrated by looking at an individual subjects treatment. For example, FIGS. 4A-4C illustrate a tacrolimus serum level and dosage information for a first patient treated in the control group (Control Patient); FIGS. 5A-5C illustrate a tacrolimus serum level and dosage information for a first patient treated in the PPM group in accordance with Function (2) (First PPM Patient); and FIGS. 6A-6B illustrate a tacrolimus serum level and dosage information for a second patient treated in the PPM group in accordance with Function (1) (Second PPM Patient). The serum level and dosage information was determined for the twenty days following the liver transplant or until the patient was discharged from the hospital.

Table 2, reproduced below, shows a comparison of the clinical features (e.g., physical characteristics) of the three patients.

TABLE 2
Clinical features of PPM Patient and Control Patient

| | Control Patient | PPM Patient 32 | PPM Patient 28 |
|---|---|---|---|
| Gender | Female | Male | Female |
| Age | 42 | 28 | 34 |
| Weight (kg) | 61.2 | 70.9 | 55.8 |
| Height (m) | 1.664 | 1.829 | 1.676 |
| Race | White | White | White |

The Control Patient was released from the hospital 19 days after the initial liver transplant. During post-operative treatment via the conventional titration method, the tacrolimus serum level of the Control Patient displayed extended deviation from the target range. Table 3, below, shows the serum level, dosage, immunosuppressant ratio, GFR, AST, ALT and Creatinine (CREAT) levels of the Control Patient for each day.

TABLE 3
Medical Data of First Control Patient

| | Serum | | | | |
|---|---|---|---|---|---|
| | Level | Dose | Immunosuppressant | | |
| Day | (ng/mL) | (mg) | Ratio | GFR | AST/ALT/CREAT |
| 0 | <3.0 | 3 | | 6 | 46/25/7.01 |
| 1 | <3.0 | 3 | | 19 | 238/94/2.76 |
| 2 | 3.9 | 3 | 0.769231 | 40 | 83/47/1.45 |
| 3 | 4 | 4 | 1 | 24 | 41/27/2.23 |
| 4 | 6.4 | 5 | 0.78125 | 25 | 39/31/2.15 |
| 5 | 9.6 | 5 | 0.520833 | 43 | 21/25/1.34 |
| 6 | 7.9 | 3 | 0.379747 | 59 | 39/35/0.93 |
| 7 | 9.1 | 4 | 0.43956 | 59 | 83/87/0.8 |
| 8 | 11.2 | 4 | 0.357143 | 59 | 37/64/0.75 |
| 9 | 18.4 | 3.5 | 0.190217 | 59 | 37/53/0.92 |
| 10 | 20.2 | 0 | 0 | 58 | 36/49/1.04 |
| 11 | 19.2 | 0 | 0 | 58 | 41/44/1.04 |
| 12 | 13.9 | 0 | 0 | 43 | 78/61/1.36 |
| 13 | 9.5 | 2 | 0.210526 | 51 | 26/52/1.16 |
| 14 | 9.2 | 2 | 0.217391 | 59 | 16/34/1.01 |
| 15 | 7.5 | 2 | 0.266667 | 59 | 14/27/0.82 |
| 16 | 7.9 | 3 | 0.379747 | 59 | 15/20/0.8 |
| 17 | 6.7 | 3 | 0.447761 | 59 | 19/24/0.88 |
| 18 | 5.6 | 3 | 0.535714 | 59 | 16/20/0.71 |
| 19 | 8.2 | 4.5 | 0.54878 | 59 | 14/16/0.73 |

The data displayed in Table 3, above, is illustrated in FIGS. 4A-4C. FIG. 4A shows a chart 410 depicting the tacrolimus serum level for the Control Patient, FIG. 4B shows a chart 420 depicting the tacrolimus dosage given to the Control Patient, and FIG. 4C shows a chart 430 depicting the immunosuppressant ratio of the Control Patient.

As shown, the tacrolimus serum level of day 1 was much lower than the target range between 8 and 10 ng/mL. The dosage was gradually increased to day 5, when the tacrolimus serum level was just below 10 ng/mL, within target range 412. After day 5, the dosage was decreased in an attempt to maintain the tacrolimus serum level within target range 412. On day 6, the tacrolimus serum level fell just below 8 ng/mL and, after slight increase of dosage of days 7 and 8, the serum level became higher than 10 ng/ml. From days 9 to day 12, the serum level stayed well above the target range despite the decreasing the dosage. During this period, the immunosuppressant ratio decreased linearly with time, likely due to the fact the GFR started to drop below the normal value to 43 at day 12 before increasing. The increasing conversion rate from dose to serum level was faster than the decreasing rate of the dosage. After day 12, the dose increased, but the serum level dropped below target range 412 as the immunosuppressant ratio rapidly increased. At day 19, the serum level was back within the target range. At this point, the doctors determined the biological markers were steady enough for the Control Patient to be released from the hospital.

On the other hand, First PPM Patient was released from the hospital 12 days after the initial liver transplant. Table 4, reproduced below, shows the serum level, dosage, immunosuppressant ratio, GFR, AST, ALT and Creatinine levels for the First PPM Patient each day.

TABLE 4
Medical Data of First PPM Patient

| | Serum | | | | |
|---|---|---|---|---|---|
| | Level | Dose | Immunosuppressant | | |
| Day | (ng/mL) | (mg) | Ratio | GFR | AST/ALT/CREAT |
| 0 | 3.6 | 1 | 0.26 | 39 | 250/108/3.1 |
| 1 | | 0 | | 44 | 877/469/2.81 |
| 2 | | 0 | | 55 | 202/270/1.84 |
| 3 | | 0 | | 55 | 129/208/1.07 |
| 4 | <3 | 2 | | 27 | 110/197/1.8 |
| 5 | <3 | 4 | | 28 | 37/148/1.77 |
| 6 | 8 | 8 | 1.0 | 28 | 43/146/1.66 |
| 7 | 13.5 | 6 | 0.444 | 49 | 32/126/1.68 |
| 8 | 7.7 | 4 | 0.519 | 44 | 18/96/1.84 |
| 9 | 8.2 | 4.5 | 0.549 | 45 | 34//84/1.82 |
| 10 | 9.4 | 5 | 0.532 | 37 | 28/76/2.15 |
| 11 | 3.6 | 5 | 1.39 | 49 | 17/59/2.14 |

The data displayed in Table 4, above, is illustrated in FIGS. 5A-5C. FIG. 5A shows a chart 510 depicting the tacrolimus serum level for the First PPM Patient, FIG. 5B shows a chart 520 depicting the tacrolimus dosage given to the First PPM Patient, and FIG. 5C shows a chart 530 depicting the immunosuppressant ratio of the First PPM Patient.

As shown, the serum level on the day of surgery was lower than the target level. During days 1-3, no tacrolimus was given to the patient. On day 4, 2 mg of tacrolimus was administered to the First PPM Patient based on the immunosuppressant ratio of day 1. The serum level was then tested and was found to be below the detection limit (<3 ng/ml). The dose was increased to 4 mg on day 5 and the serum level was still below detection levels. The dosage was then doubled on day 6 and the serum level reached target range 512 at 8 ng/mL. The dosage was then dropped after the serum level exceeded target range 512 and the immunosuppressant ratio decreased. Based on the immunosuppressant ratios, the dosage was then calculated for day 8. As shown, both the immunosuppressant ratio and the serum level were kept relatively constant from days 8-10 and the serum level was kept near or within target range 512 for days 8, 9, and 10. Although the serum level of day 11 dropped substantially despite the dosage being the same as the previous day, this drop was likely do to the discontinuation of cefepime, metronidazole and sulfamethoxazole on day 11. The patient was discharged on day 12. The First PPM Patient illustrates the complexities of tacrolimus dosing as it is common to have gaps in some of the necessary data. By utilizing the immunosuppressant ratio the current methods can calculate dosages without the need for three consecutive days of stable patient data, as is required by other formula. To illustrate this, a dosage for the First PPM Patient was able to be determined on day 8, where other formulas would not have been available to determine dosage until day 10.

Referring now to charts 610 and 620 of FIGS. 6A and 6B, respectively, the serum level and immunosuppressant ratio the Second PPM Patient are shown. As show in chart 610, the immunosuppressant ratio of the Second PPM Patient slightly overshoots target range 612 on day 5. The serum level is then maintained near or within target range 612 for days 6-10. The Second PPM Patient was released on day 11.

As shown above for the Control Patient, the First PPM Patient, and the Second PPM Patient, the changes to the liver (AST/ALT) and kidney (GFR) functions will cause the serum level to change, typically outside of the target range. The present systems, as described above, can bring the serum level back to the target range within one or two days. Comparatively, as shown in FIG. 4A, conventional treatment takes several days (six or more) to bring the serum level back within the target range. The improved adjustment time of the present systems allows for faster recovery and quicker discharge times.

Additionally, data from the clinical trial was utilized to determine the average deviations between a target (i.e., predicted) serum level and an actual (i.e., measured) serum level for the two PPM patients. Table 5, reproduced below shows the average deviations using calculations from thee different functions: Function 0, Functions 1, and Function 2.

TABLE 5
Serum Level Deviations

| | Function 0 | Function 1 | Function 2 |
|---|---|---|---|
| Average Deviation from | 2.9 | 2.0 | 1.4 |
| Target Serum Level | | | |
| Average Deviation from | 1.6 | 1.2 | 0.6 |
| Target Serum Level | | | |

Where Function 0 is S_{T}(D_{T}, t)=x₀(t)+x₁(t)D_{T}+x₁₁(t)D_{T}²; Function 1 is [(D/S)T]_{(N+1) day}=[(D/S)_{T}]_{N day}; and Function 2 is [(D/S)T]_{next day}=x₀(t)+x₁(t)[AST×(D/S)_{T}]_{today}+z11(t)[AST×(D/S)T] today. Table 5 shows that the deviations from the target serum levels are smaller when using the functions (e.g., Functions 1 and 2) of the present systems and methods. By incorporating the AST level the current system and methods can provide further improvement in achieving a target serum levels

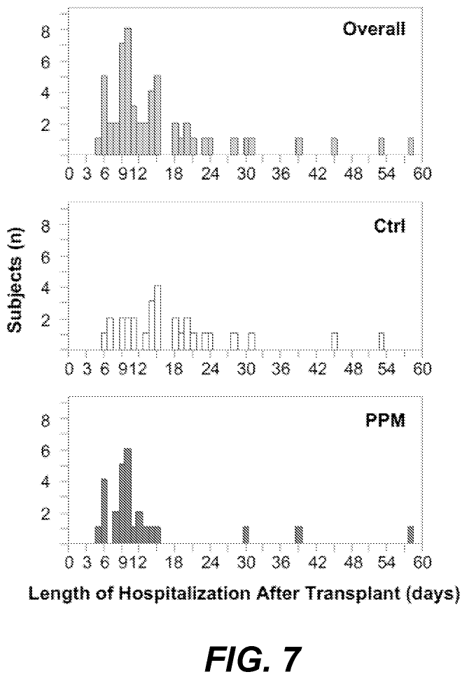

Turning now to the entire population of the clinical trial, we can see the PPM based personalized dosing of the PPM group outperformed standard-of-care physician-guided dosing for the Control group. For example, referring now to FIG. 7, the number of days a patient spent in the hospital before being released is shown for the Control group (Ctrl), the PPM group, and the overall population. The patients in the PPM group tended to have a shorter stay in the hospital than the Control group. For the PPM group 90% of the patients were discharged by day 15. The discharge time for the Control group was much longer with 90% of the patients not being discharged until day 25. As shown, almost all subjects in the PPM group were released by the seventeenth day following transplant. In all, patients in the Control group had a median length-of-stay (LOS) of 15 days (Q1-Q3 10.5-20.5) and patients in the PPM group had a median LOS of 10 days (Q1-Q3 8-12), with a p-value of 0.0026. Additionally, the average hospital stay was 15.87 day for the Control group and 10.42 days for the PPM group. As shown in FIG. 7, each group appears to have at least one outlier that required a much longer hospital stay than the average patient. To remove such outliers, the average hospital stay was also calculated removing the data from the two patients with the longest time in the hospital. After the removal, the average hospital stay was 14.36 days for the Control group and 8 days for the PPM group.

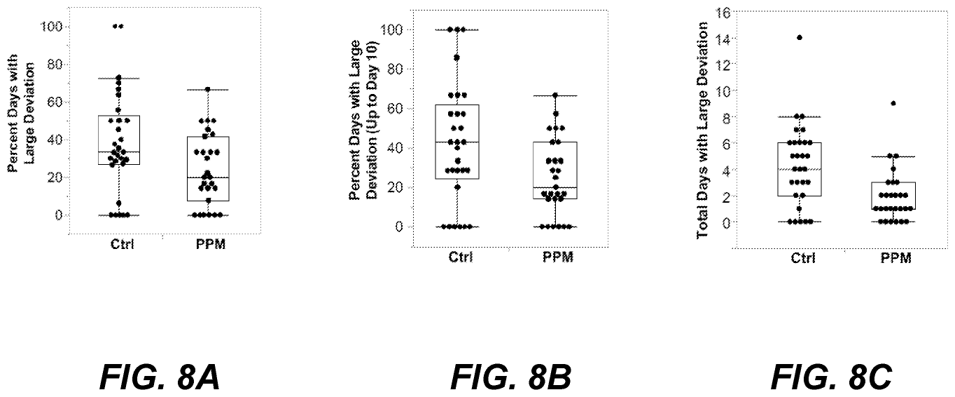

The primary outcome measure of the clinical trial was percentage of days with large blood tacrolimus trough level deviations from target range (8-10 ng tacrolimus/mL blood). Large deviations from the target range were defined as a greater than 2 ng/ml deviation outside of the target range. As shown in FIGS. 8A-8C, the subjects in the PPG group experienced less large deviations than that of the Control group. For example, as illustrated in FIG. 8A, a percentage of post-transplant days with large deviations from target range was higher in the Control group than in the PPM group. The average percentage of post-transplant days with large deviations from target range was 38.4±27.4% for the control group and 24.2±19.1% for the PPM Group, with a p-value of 0.029. As shown in FIG. 8B, a comparison of the percentage of post-transplant days with large deviations between the PPM Group and the Control group during the first 10 days was performed. By limiting the time period to the first 10 days, the effect of the length of stay at the hospital can be minimized and focus on the early post-transplant period, during which tacrolimus dosing is most labile. During the first ten day time period, patients in the control group had a mean of 42.1±30.9% of post-transplant days with large deviations from target range; the PPM group had 24.9±19.5% of post-transplant days with large deviations, with a p-value of 0.015. Further, as shown in FIG. 8C, the total number of post-transplant days with large deviations was also significantly different between the two groups. The Control group had a median of 4 days of large deviations (Q1-Q3 2-6) and the PPM group had a median of 1 day of large deviations (Q1-Q3 1-3), with a p-value of 0.0023.

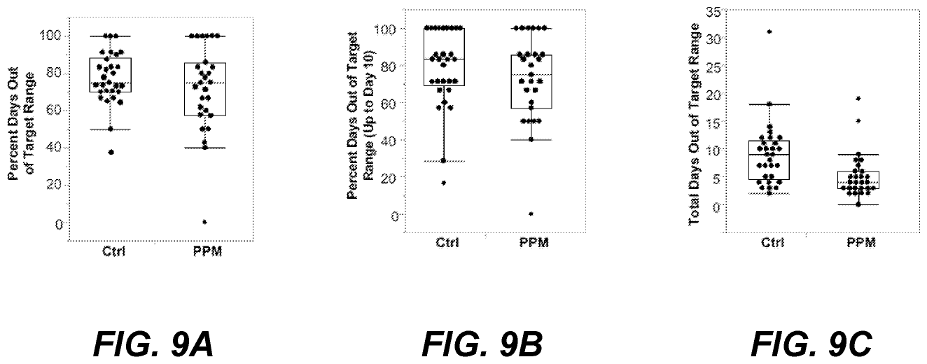

Referring now to FIGS. 9A-9C, comparisons between the percentage of days the subjects were outside-of-target-range for the Control group and the PPM group are shown. The differences in the total deviations were less significant than the large deviations of FIGS. 8A-8C. As shown in FIG. 9A, patients in the Control group had an average percentage of 77.3±14.2% of post-transplant days with blood tacrolimus trough level outside the target range and the PPM group had an average percentage of 71.7±23.2% of post-transplant days outside the target range, with a p-value of 0.30. Limiting the observation window to the first ten days shows similar results, where the PPM group showed only a slight decrease in the number of days with blood tacrolimus trough level outside the target range. FIG. 9B shows that, during the first ten days post-transplant, the patients in the Control group had an average percentage of 78.6±21.1% of post-transplant days outside of the target range and the PPM group had an average percentage of 72.9±23.5% of post-transplant days outside of the target range, with a p-value of 0.35. However, as shown in FIG. 9C, the total number of post-transplant days outside of the target range was significantly lower in the PPM group, at a median of 4 days (Q1-Q3 3-6) outside of the target range, than in the Control group, which had a median of 9 days (Q1-Q3 4.5-11.5) outside of the target range, with a p-value of 0.0014. This result was likely affected by the finding that the PPM group patients were discharged earlier.

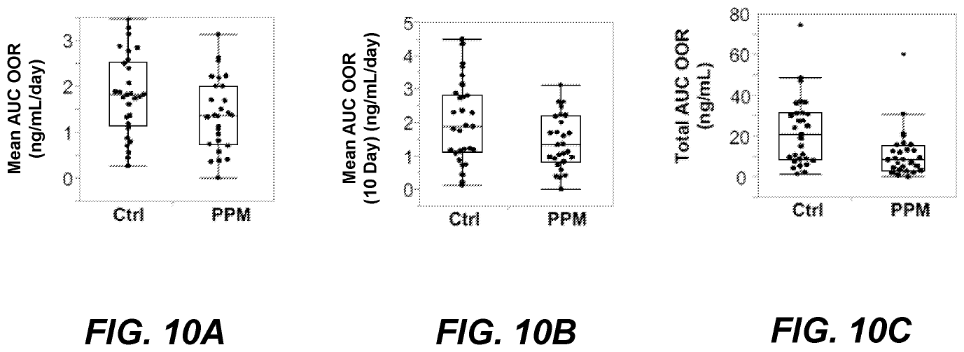

The overall average AUC for the Control group and the PPM group is shown in FIG. 10A and the first ten day average AUC is shown in FIG. 10B. While the difference between the average area-under-the-curve (AUC) outside-of-target-range between the Control group and the PPM group did not reach statistical significance overall (Control: 1.81±0.88 versus PPM: 1.40±0.79 ng/ml/day; P=0.069), it was statistically lower in the PPM group within the first ten days (Control: 2.01±1.22 versus PPM: 1.41±0.81 ng/ml/day; P=0.035). The overall average AUC for the Control group and the PPM group is shown in FIG. 10A and the first ten day average AUC is shown in FIG. 10B. As illustrated in FIG. 10C, the total AUC outside-of-target-range was also much lower in the PPM group than the Control group (Control: 22.1±16.7 versus PPM: 11.3±12.1 ng/mL; P=0.0062).

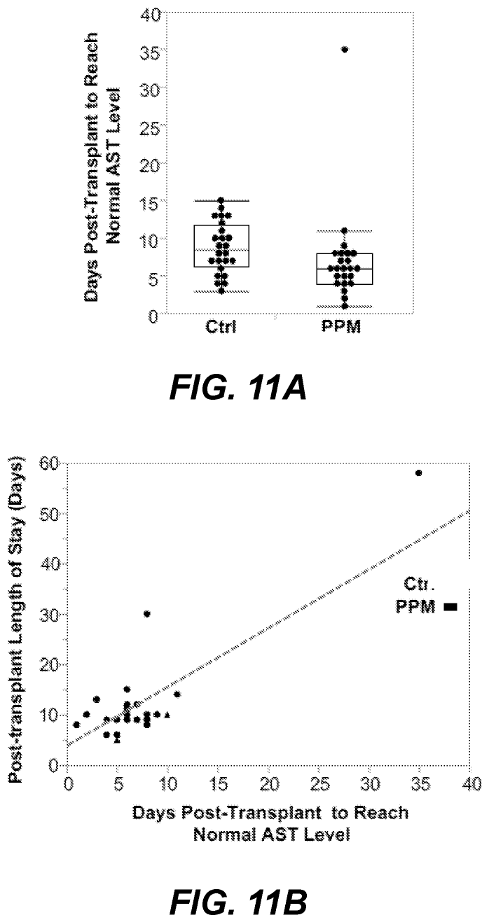

Further, to understand potential reasons underlying the large difference in the length-of-stay (LOS) between the Control group and the PPM group, the number of days it took for the aspartate aminotransferase (AST) to normalize (termed DAST) was analyzed. As shown in FIG. 11A, it was found that the PPM group reached normal AST levels more quickly than the SOC group. Specifically, patients the control (Ctrl) group took a median of 8.5 days (Q1-Q3 6.25-11.75) to normalize AST levels, whereas the patients in the PPM group reached normal AST levels at 6 days (Q1-Q3 4-8) (P=0.014). The LOS and days to normalize AST levels (DAST) were analyzed for the PPM group subject. As shown in FIG. 11B, the LOS was also found to be have a moderate direct correlation with DAST of the PPM group, with coefficient of determination (R²) being equal to 0.45. Six patients, three in each group, whose AST levels did not reach normal levels prior to discharge from the hospital, were excluded from the coefficient of determination analysis.

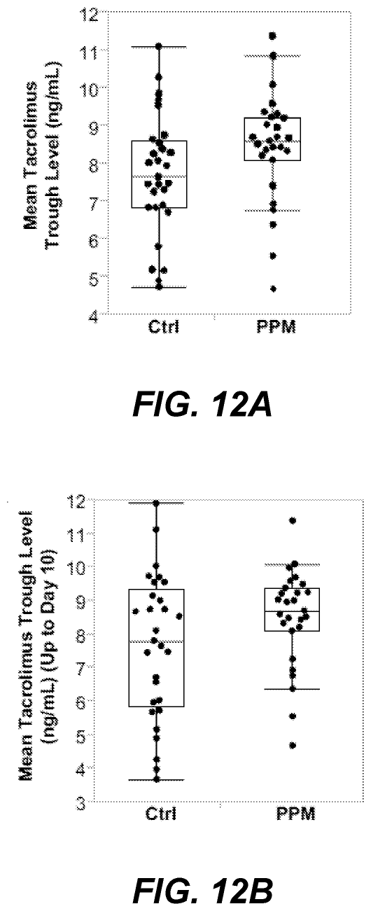

Referring now to FIGS. 12A and 12B, the average blood tacrolimus trough levels for the Control group and the PPM group are shown for the entire hospital stay (FIG. 12A) and for the first ten day (FIG. 12B). The mean tacrolimus trough level in the PPM group was higher than the Control group both throughout the hospital stay (PPM: 8.4±1.5 versus Control: 7.7±1.6 ng/ml respectively; P=0.037) and within the first ten days (PPM: 8.5±1.5 versus Control: 7.6±2.2 ng/ml respectively; P=0.088). Additionally, the average blood tacrolimus trough level in the PPM group was found to be within the target range of 8-10 ng/mL.

As explained above, the PPM based personalized dosing outperformed standard-of-care physician-guided dosing by decreasing the rate of large deviations from the target blood trough range in daily dosing of tacrolimus after liver and liver/kidney transplantation. PPM dosed patients have a 37% decrease in the percentage of days with a large deviation from the target trough drug level. Such a reduction of the magnitude of intra-patient trough level variability, as well as the period of time in which large variations occur can lead to a tremendous impact on post liver transplant outcomes. The decrease in the percentage of days with a large deviation from target levels and a 30% reduction of the mean AUC outside of target range achieve by PPM are a significant step in the direction of improving graft and patient outcomes in liver transplantation. Furthermore, the PPM system and methods were able to start dosing patients very early in the post-transplant period, decreasing the typical run-in time of ten days to only three days.

In addition to potentially improving long-term outcomes, the PPM system, described herein, has the potential to reduce cost and improve short-term outcomes. Patients in the PPM group were discharged from the hospital a median of five days earlier after transplantation than the Control group, a more than 30% reduction in the post-transplant length of stay. One potential explanation of how better dosing can lead to shorter length of stay is hinted at by the additional finding that PPM group patients had a shorter time to normalization of AST, as there is a linear correlation between the POD at which the AST becomes normal and the overall length of stay, as shown in FIG. 11B

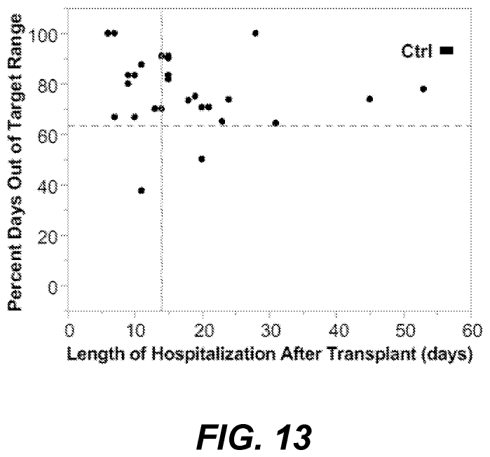

It is possible that better dosing can lead to improved liver function, especially in the early post-transplant period (e.g., first ten days post-surgery). There is also a tendency towards higher average tacrolimus levels and PPM dosing. The mean tacrolimus level was shown to be higher in PPM group overall, and it is significantly higher when examining just the first 10 days after transplant. Additionally, the standard deviation of the tacrolimus levels is smaller indicating less variability in dosing. One can speculate that better immunosuppression with higher tacrolimus levels in the early post-transplant period leads to improved liver enzyme levels. For example, only 6.9% (2/29) of the Control group had tacrolimus levels out of target range less than 62% of the time compared to 33.3% (9/27) of the PPM group (P=0.018). Stated another way, only two subjects in the Control group had their tacrolimus levels within the target range for more than 38% of the post-transplant days (FIG. 13). Yet, the PPM group had nine subjects that maintained tacrolimus levels within the target range for more than 38% of the post-transplant days, despite having a smaller population. Additionally, only 18.5% (5/27) of the PPM group had a post-transplant length of stay two weeks or more versus 65.5% of the Control group (P=0.0005). FIG. 13 depicts a correlation of tacrolimus levels out of target range with post-transplant length of stay for the Control group. As shown in FIG. 13, the horizontal dashed line represents 62% of days outside the target range and the vertical (dotted) line represents 2 weeks.

Thus, the present systems and methods can lead to improved tacrolimus administration without the need for mechanistic information by implicitly accounting for genomic, pharmacokinetic, and other patient factors to produce a dosing recommendation. The systems and methods described herein, is applicable and valuable in applications of varying complexity, such the above trial, where PPM repeatedly overcame a substantial set of hurdles presented by the complex biological, physiological, and clinical context of post-transplant tacrolimus dosing. However, the present systems and methods are not limited to tacrolimus dosing and would provide a straightforward and attractive solution for handling human diversity in drug dosing needs for a variety of other clinical problems, such as overall immunomodulation or cancer therapy.

The above specification and examples provide a complete description of the structure and use of illustrative implementations. Although certain implementations have been described above with a certain degree of particularity, or with reference to one or more individual implementations, those skilled in the art could make numerous alterations to the disclosed implementations without departing from the scope of this invention. As such, the various illustrative implementations of the methods and systems are not intended to be limited to the particular forms disclosed. Rather, they include all modifications and alternatives falling within the scope of the claims, and implementations other than the one shown may include some or all of the features of the depicted implementation. For example, elements may be omitted or combined as a unitary structure, or connections may be substituted. Further, where appropriate, aspects of any of the examples described above may be combined with aspects of any of the other examples described to form further examples having comparable or different properties or functions, and addressing the same or different problems. Similarly, it will be understood that the benefits and advantages described above may relate to one implementation or may relate to several implementations.

The claims are not intended to include, and should not be interpreted to include, means-plus- or step-plus-function limitations, unless such a limitation is explicitly recited in a given claim using the phrase(s) “means for” or “step for,” respectively.

1. A method for determining an immunosuppressant dosage for a patient after an organ transplant, the method comprising:
obtaining a first dosage of an immunosuppressant drug administered to a patient on a first time period;
obtaining a first serum level of the patient associated with the first time period;
determining a second dosage of the immunosuppressant drug based on a first immunosuppressant ratio between the first dosage and the first serum level;
generating a first output based on the second dosage;
wherein:
the second dosage associated with a second time period; and
the first time period immediately precedes the second time period.

2. The method of claim 1, further comprising:
obtaining a target range associated with a target serum level; and
wherein determining the second dosage of the immunosuppressant drug is based on the target serum level.

3. The method of claim 2, wherein:
the first immunosuppressant ratio is a ratio of the first dosage to the first serum level; and
determining the second dosage includes multiplying the first immunosuppressant ratio by the target serum level.

4. The method of claim 1, wherein the second dosage of the immunosuppressant drug is calculated independent of an aspartate transaminase (AST) level, an alanine transaminase (ALT) level, a glomerular filtration rate (GFR), or a combination thereof.

5. The method of claim 1, further comprising:
obtaining a second serum level of the patient associated with the second time period;
determining a third dosage of the immunosuppressant drug based on a second immunosuppressant ratio between the second dosage and the second serum level;
obtaining a third serum level of the patient associated with a third time period;
determining a fourth dosage of the immunosuppressant drug based on a third immunosuppressant ratio between the third dosage and the third serum level; and
generating a second output based on the fourth dosage, the third dosage associated with a fourth time period;
wherein:
the second time period precedes the third time period; and
the third time period precedes the fourth time period.

6. The method of claim 5, further comprising:
obtaining an first aspartate transaminase (AST) level of the patient associated with the first time period;
obtaining an second AST level of the patient associated with the second time period;
obtaining an third AST level of the patient associated with the third time period; and
based on the first AST level, the second AST level, the third AST level, or a combination thereof, being within a threshold range, determining serum coefficients.

7. The method of claim 6, further comprising:
determining a fourth dosage of the immunosuppressant drug based on the serum coefficients and a ratio between the third dosage and a third serum level.

8. The method of claim 1, wherein the first time period is a first twenty-four hour period and the second time period is a second twenty-four hour period that immediately follows the first twenty-four hour period and wherein the first serum level of the patient associated with the first time period is an amount of the immunosuppressant drug in the blood stream of the patient during a time within the first twenty-four hour period.

9-10. (canceled)

11. The method of claim 1, wherein the first time period is within three days following an organ transplant operation.

12. (canceled)

13. The method of claim 1, wherein the first serum level includes an amount of immunosuppressant drug in the patient's blood stream.

14-24. (canceled)

25. The method of claim 5, wherein the second output is generated using the function:
$${[ {( D / S )}_{T} ]}_{next day} = {x}_{0} ( t ) + {{x}_{1} ( t ) [ AST \times {( D / S )}_{T} ]}_{today} + {{z}_{11} ( t ) [ AST \times {( D / S )}_{T} ]}_{today}$$

26. (canceled)

27. A variable dosing system comprising:
a memory storing one or more instructions; and
a processor coupled to the memory and configured to execute the one or more instructions to:
determine a first dosage of an immunosuppressant drug given to a patient on a first day;
determine a first serum level of the patient associated with the first day;
calculate a second dosage of the immunosuppressant drug based on a first ratio of the first dosage to the first serum level; and
initiate a first output associated with the second dosage.

28. The variable dosing system of claim 27, wherein the one or more instructions, when executed by the processor, further cause the processor to:
receive an input associated with a target serum level; and
calculate the second dosage by multiplying the first ratio by the target serum level.

29. The variable dosing system of claim 27, wherein the one or more instructions, when executed by the processor, further cause the processor to:
store the first ratio;
store the second dosage;
determine a second serum level of the patient associated with a second day;
determine a third serum level of the patient associated with a third day;
calculate a fourth dosage of the immunosuppressant drug based on:
a second ratio of the second dosage to the second serum level; and
a third ratio of a third dosage to the third serum level; and
initiate a second output associated with the fourth dosage.

30. The variable dosing system of claim 29, wherein the first day immediately precedes the second day.

31. The variable dosing system of claim 27, wherein determining the first serum level comprises:
receiving, from a medical testing device, medical data associated with the patient on the first day; and
identifying the first serum level from the medical data.

32. The variable dosing system of claim 27, further comprising:
a display device; and
wherein the first output is displayed on the display device.

33. The variable dosing system of claim 29, wherein the one or more instructions, when executed by the processor, further cause the processor to:
determine an first AST/ALT level of the patient associated with the first day;
determine an second AST/ALT level of the patient associated with the second day;
determine a third AST/ALT level of the patient associated with a third day; and
based on each of the AST/ALT levels being within a threshold range, calculating serum coefficients.

34. The variable dosing system of claim 33, wherein the one or more instructions, when executed by the processor, further cause the processor to:
determine a third serum level of the patient associated with the third day; and
calculate a fourth dosage of the immunosuppressant drug based on a ratio of a third dosage to the third serum level, the third AST/ALT level, and the serum coefficients.

35. The variable dosing system of claim 33, wherein each of the first, second, and third AST/ALT levels are within 10% of each other when the AST/ALT levels are within the threshold range.

36. (canceled)
